(19) United States
(12) Patent Application Publication
(10) Pub. No.: US 2011/0011261 A1
(43) Pub. Date: Jan. 20, 2011

(54) WET SCRUBBER FOR CARBON DIOXIDE COLLECTION

(75) Inventors: Blair L. Jenness, Wayzata, MN (US); Susan Jenness, Wayzata, MN (US); Donovan D. Bohn, Forest Lake, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(73) Assignee: TRIANGLE ENERGY GROUP, LLC, Coon Rapids, MN (US)

(21) Appl. No.: 12/838,640

(22) Filed: Jul. 19, 2010

Related U.S. Application Data
(60) Provisional application No. 61/226,515, filed on Jul. 17, 2009.

(51) Int. Cl. B01D 53/14; B01D 47/00; C12M 1/107

(52) U.S. Cl. 95/150; 261/110; 435/300.1

(57) ABSTRACT

A scrubber unit for removal of carbon dioxide from flue gas, including a first chamber, a second chamber, a partition dividing the first chamber and the second chamber, a first weir to distribute flow across of aqueous absorbent across the chamber, and a second weir in the second chamber. The partition is positioned to allow fluid to flow from the first chamber to the second chamber beneath a lower edge of the partition. The scrubber may be used in an algae growing system, along with an algae bioreactor, with the aqueous absorbent solution flowing in a continuous circuit through the wet scrubber and the algae bioreactor to absorb carbon dioxide in the wet scrubber and to provide the absorbed carbon dioxide to algae.

### PRIORITY CLAIM

The present application claims priority to U.S. provisional patent application No. 61/226,515 filed Jul. 17, 2009, the disclosure of which is hereby incorporated by reference in its entirety.

### FIELD OF THE INVENTION

The subject of this application relates generally to systems and methods for removal of target pollutants from gaseous streams, and more particularly to a wet scrubber system for removal of carbon dioxide from combustion gases.

### BACKGROUND

Air quality control systems may include scrubber systems for reducing the level of certain pollutants in air emissions. These scrubber systems have been developed for controlling harmful emissions from industrial and utility boilers like those used in power plants. Such scrubber systems are generally of either the dry or wet types. Dry scrubber systems generally include an open chamber in which the flue gas is directed through a liquid spray of lime and fly ash slurry. A reaction occurs with the sulfur dioxide in the gas to form a calcium compound in dry particulate form which can then be collected at the outlet of the chamber, thereby “scrubbing” the flue gas free of sulfur dioxide pollutants.

On the other hand, in the so-called “wet scrubbers” sulfur dioxide is collected in the form of a slurry in a tank of aqueous absorbent for periodic removal in a liquid slurry form. In order to avoid excessive maintenance costs from the build-up of deposits of fly ash, the fly ash must first be removed by means of filtration or electrostatic precipitation before the flue gas can be treated in a wet scrubber. As a result, such systems require the use of two large and expensive structures, one for the removal of fly ash and the other for the removal of sulfur dioxide. Further, since the fly ash is typically removed separately, the alkalinity which otherwise would have been added by the presence of fly ash is not present, and thus some of the fly ash which was removed must then be added to the aqueous absorbent to increase the alkalinity of the slurry, which adds further expense and complication.

Air quality control systems in older power plants may consist of only particulate collectors such as fabric filters or dry electrostatic precipitators followed by a sulfur dioxide scrubber. The scrubber may be either of the wet or dry type. In contrast, newer air quality control systems may be much more sophisticated. These systems may include ammonia injection followed by selective catalytic reduction to remove nitrogen oxides, fabric filter or dry electrostatic precipitator to remove ash, lime injection to remove sulfur trioxide formed in a selective catalytic reduction step, activated carbon injection to remove mercury, another fabric filter to remove the activated carbon and lime, a wet limestone scrubber or dry scrubber to remove sulfur dioxide, and a wet electrostatic precipitator to remove other fine particulate or droplets of mercury. While such systems remove a large portion of the pollutants from the power plant emissions, they do not remove carbon dioxide (CO₂) to a significant degree.

The reduction of CO₂ emissions is of increasing environmental importance, due to the role of CO₂ as a “greenhouse gas.” For example, pending legislation could force many cement plants to close. However, even if CO₂ were removed from flue gas, the disposal of such large quantities of CO₂ presents a significant problem. While the gas could be stored under ground, such disposal is risky because if such stored gas should escape, it would be extremely hazardous.

In addition to CO₂ emissions, the large amount of water consumed by power plants also is an environmental concern. Traditionally, power plants are sited at locations where adequate water is available for use primarily by the evaporative cooling towers and wet or dry scrubbers. The scrubber systems add water vapor to the flue gas exiting the boiler, and this water is then lost to the atmosphere. Existing power plants incorporate evaporative cooling towers as a convenient means to discharge heat to the atmosphere. For example, at a typical 600 MW power plant operating at 80% capacity factor, the water consumption can exceed 5,000 acre feet per year.

Further improvement is therefore needed in existing air quality systems to provide for removal of CO₂ and to reduce the consumption and loss of water.

### FIGURES

The following drawings are illustrative of particular embodiments of the present invention and therefore do not limit the scope of the invention. The drawings are not to scale (unless so stated) and are intended for use in conjunction with the explanations in the following detailed description. Embodiments of the present invention will hereinafter be described in conjunction with the appended drawings, wherein like numerals denote like elements.

FIG. 1 is a partial schematic view of a power plant including air quality elements for removing pollutants according to embodiments of the invention;

FIG. 2 is a vertical cross-sectional schematic view of a CO₂ scrubber module according to embodiments of the present invention;

FIG. 3 is a partial perspective view of a vertical partition located in a CO₂ scrubber module according to embodiments of the invention; and

FIG. 4 is a flow chart of a CO₂ scrubber module according to an embodiment of the invention in an algae production system.

### SUMMARY

Embodiments of the invention include scrubber units for removal of CO₂ from flue gas. In some embodiments, the scrubber unit includes a first chamber having a gas inlet and a fluid inlet, a second chamber having a gas outlet and a fluid outlet and a partition dividing the first chamber and the second chamber. The partition is positioned to allow fluid to flow from the first chamber to the second chamber beneath a lower edge of the partition. In some embodiments, the lower edge of the partition may form a zigzag. The scrubber unit also includes a first weir to distribute the flow of fluid across the chamber, and a second fluid level control, such as a weir, in the second chamber. The first weir may extend across the width of the first chamber such that fluid entering the first chamber through the fluid inlet must flow over the first weir to pass through the scrubber. The second level control may be a weir sized such that a desired fluid level is maintained between the partition and the second weir. In some embodiments, the scrubber may remove between about 70 and 99% of CO₂ from the flue gas.

An aqueous absorbent may flow through the scrubber, entering the scrubber through the fluid inlet and exiting through the fluid outlet. In some embodiments, the aqueous absorbent may be a carbonate salt and a bicarbonate salt, such as a solution of sodium carbonate and sodium bicarbonate. In some embodiments, the aqueous absorbent may have a pH between about 9 and 11. In other embodiments, the aqueous absorbent may have a pH between about 9.5 and 10.5.

In some embodiments, the wet scrubber for the removal of carbon dioxide from flue gas may be a component of an algae growing system. The algae growing system may include a CO₂ scrubber, an aqueous absorbent salt solution, and an algae bioreactor. The aqueous absorbent solution may flow in a continuous circuit through the wet scrubber and the algae bioreactor to absorb CO₂ in the wet scrubber and to provide the absorbed CO₂ to algae. The aqueous absorbent may be a solution of a carbonate salt and a bicarbonate salt, such as sodium carbonate and sodium bicarbonate.

The algae growing system may grow one or more species of algae in the aqueous absorbent solution, such as one or more species of Spirulina. In some embodiments, the algae growing system also includes a heat exchanger. In such embodiments, the aqueous absorbent may flow from the scrubber to the heat exchanger, and then from the heat exchanger to the algae bioreactor.

Other embodiments include methods of removing CO₂ from flue gas. In some embodiments, the method includes passing flue gas through a gas inlet into first chamber of a scrubber, the first chamber including a fluid inlet and an aqueous absorbent in a lower portion of the first chamber, bubbling the flue gas into the aqueous absorbent, flowing the aqueous absorbent including the flue gas bubbles into a lower portion of a second chamber of the scrubber, releasing the flue gas bubbles as clean flue gas from the aqueous absorbent and into the second chamber, and passing the gas through a gas outlet of the second chamber. The aqueous absorbent may be a carbonate salt and a bicarbonate salt, such as sodium carbonate and sodium bicarbonate. In some embodiments, the first chamber includes a first weir, and the method also includes flowing the aqueous absorbent over a first weir to distribute the aqueous absorbent across the width of the first chamber. In some embodiments, the second chamber includes a second weir, and the method includes controlling the depth of the aqueous absorbent between the partition and the second weir. In some embodiments, the method includes removing between about 70 and 99% of the CO₂ from the flue gas.

### DETAILED DESCRIPTION

The following detailed description is exemplary in nature and is not intended to limit the scope, applicability, or configuration of the invention in any way. Rather, the following description provides practical illustrations for implementing exemplary embodiments of the invention.

In an embodiment of the invention, Applicants provide a bubbler scrubber or scrubber unit that can be used as a wet scrubber for the capture of CO₂. Sources of flue gas from which CO₂ may be removed include power plants, cement plants, and breweries, for example. Scrubber units according to the present invention may be used in newly constructed boiler systems or incorporated into existing scrubber systems as a retrofit. They may be used in addition to, or instead of, existing scrubber units or may be placed in series with other scrubber units used to collect or capture different target pollutants. In some embodiments, the CO₂ scrubber is a component of an air quality control system at a power plant. In some embodiments, the CO₂ scrubber is a component of an algae growing system, providing captured CO₂, removed from the flue gas, and heat to the algae. The carbon dioxide scrubber may be one component of an integrated system that removes carbon dioxide from power plant flue gas, transports the carbon dioxide to algae, grows algae using sunlight, harvests the algae using a filter, re-circulates the filtrate back to the scrubber, and processes the algae into biodiesel fuel and other products.

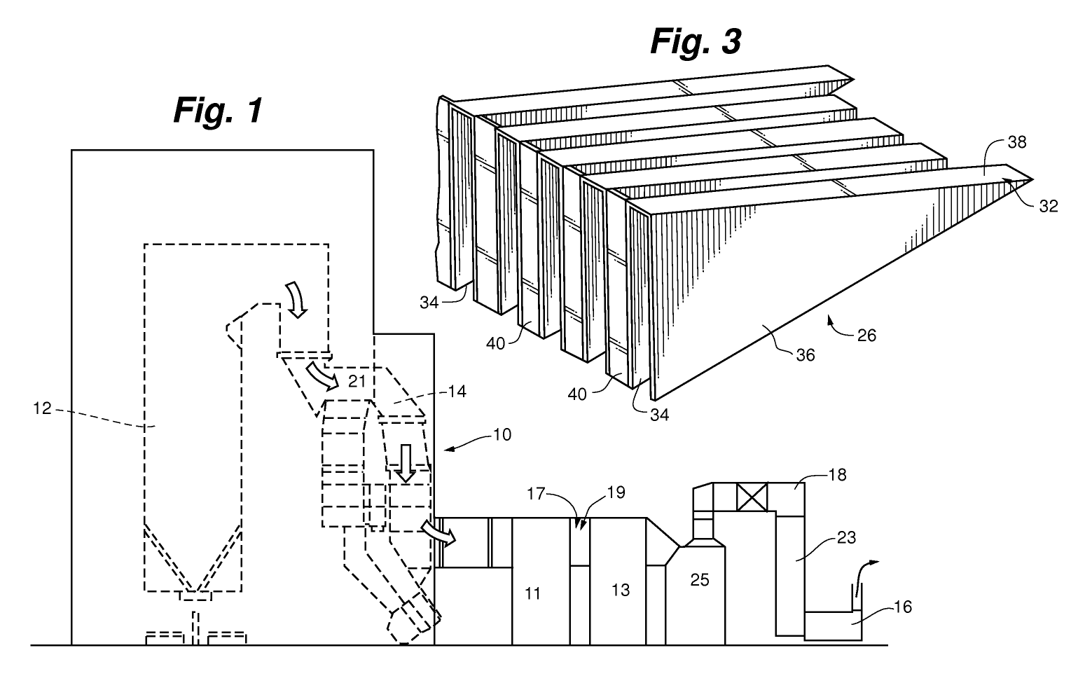

CO₂ scrubbers according to embodiments of the invention may be used as a component of a system for air quality control in a power plant. An example of such a system is shown in FIG. 1, which shows an embodiment of a CO₂ scrubber unit 16 in a power plant 10 of the type employed by utility companies for generating electricity. The power plant 10 includes a boiler 12 which generates steam for driving a turbine that turns a generator to generate electricity. The boiler 12 is fired by coal, the particular sulfur content of which depends upon the origin of the coal. Coal of low sulfur content may be used because of the lesser difficulty in meeting pollution control standards, although coal of relatively higher sulfur content is more common. The flue gas from boiler 12 is directed through duct work 14 to a series of air quality control devices including ammonia injection 21 for selective catalytic reduction, a fabric filter or dry electrostatic precipitator 11 to remove ash particulate, activated carbon injection 17 for adsorption of mercury, lime injection 19 for reaction with sulfur trioxide (created to a large extent as the flue gas passes through the selective catalytic reduction grid), a second fabric filter 13 to remove the activated carbon and lime, a wet scrubber module 25 for removal of SO₂, and a wet electrostatic precipitator to remove fine droplets 18. The flue gas passes through duct work 23 containing induced draft fans (not shown) to the CO₂ scrubber module 16, after which it may pass through additional ductwork to an exhaust stack to vent the cleaned flue gas to the atmosphere.

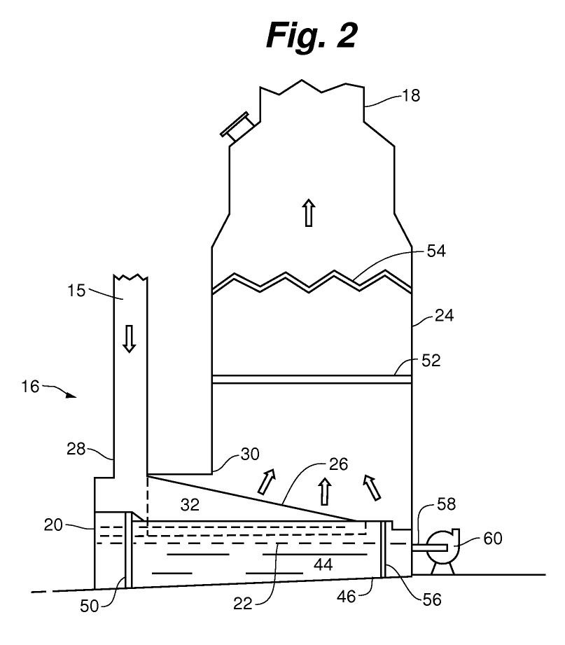

The CO₂ scrubber module 16 provides for removal of a significant portion of the carbon dioxide from the flue gas. In some embodiments, it also provides for simultaneous removal of sulfur dioxide with improved efficiency and reduced cost. Referring to FIGS. 2 and 3, there is shown the internal construction of a CO₂ scrubber module 16. The scrubber module 16 includes a vessel or tank 20 which is partially filled with aqueous absorbent 22. In some embodiments, the aqueous absorbent 22 includes water and a solution of sodium carbonate and sodium bicarbonate. A continuous stream of fresh aqueous absorbent with a high concentration of absorbent such as sodium carbonate flows through a fluid inlet, into the lower portion of the vessel 20, and over a first weir 50. The first weir 50 serves to distribute the entering stream across the width of the scrubber module 16. The reagent stream flows across the bottom of the vessel 20 and out a fluid outlet 58.

Flue gas enters the vessel 20 through duct work 15 leading from the boiler, through inlet 28 and into the vessel 20. The flue gas then enters vertical partition 26 which functions as an inverted weir, the details of which can be seen in FIGS. 3. Vertical partition 26 is located in the middle of the vessel 20, between the flue gas inlet 28 and the flue gas outlet, chamber 30. The partition 26 comprises a plurality of generally parallel fingers 32 of hollow construction arranged in a parallel, spaced apart relationship, stacked across the width of the tank 20 and extending parallel to the flow of the aqueous absorbent 22. The fingers 32 may be of a generally right triangular configuration, as shown, to reduce material cost, or they may be rectangular. Each finger 32 may be constructed of stainless steel and includes a pair of side walls 34 and 36 and an inclined top wall 38. End walls 40 are connected between adjacent edges of the open divergent ends of the fingers 32 so that all of the flue gas entering inlet 28 is directed into the hollow fingers 32 of the partition 26. In this way, the partition 26 forms a zigzag partition comprised of a number of vertical side walls 34 and 36 and connecting top walls 38 and end walls 40. In alternative embodiments, the partition may have other shapes, such as any corrugated shape having a sufficient length under the surface of the aqueous absorbent 22.

The lower edge of the partition 26 is submerged into the surface of the aqueous absorbent 22. The lower end of the vessel 20 is filled with aqueous absorbent 22 while the upper end is divided by a vertical partition 26 into two chambers, 32 and 30. Flue gas with high concentrations of carbon dioxide from the boiler and air quality control system such as described above is received in one chamber 32, while scrubbed or clean flue gas leaves the other chamber 30 on the other side of the partition for exhaust through a stack to the atmosphere.

The partition 26 forces the flue gas under the surface of the aqueous absorbent 22, creating a high liquid to gas contact, resulting in effective capture of CO₂. Specifically, the flue gas which enters the fingers 32 of the partition 26 bubbles up around the side walls 34, 36 through the liquid. This is the only pathway for the flue gas to pass through the vessel 20. As the flue gas bubbles through the liquid, carbon dioxide in the flue gas reacts with aqueous absorbent 22. In embodiments in which the flue aqueous absorbent includes sodium carbonate, the CO₂ reacts with the sodium carbonate to form sodium bicarbonate. A continuous stream of fresh liquid containing sodium carbonate, provides the primary reagent to react with the carbon dioxide to form a sodium bicarbonate which is then removed and used as a growing medium for algae production. After bubbling beneath the partition 26, the flue gas exits the vessel 26 through the gas outlet, chamber 30. The scrubbed flue gas enters outlet, chamber 30, at about 95 degrees F., passing upwardly in tower 24 first through an optional conventional bulk entrainment separator 52 and then through an optional conventional mist eliminator 54 to remove excess moisture.

Alternatively, other wet scrubbers such as those used for removal of SO₂ may be modified for use in removing CO₂ as described herein. However, such scrubbers may not work as efficiently, due to the greatly increased power to operate the recycle pumps for scrubbers that require spraying the solution through the flue gas. In other embodiments, the CO₂ scrubber may remove CO₂ by sparging the flue gas under the aqueous absorbent 22, such as by using a system of vertical, open ended pipes that pass through the floor of the scrubber. In such embodiments, flue gas may pass down through the pipes and exit at the bottom end of the pipes, such that the flue gas flows around the edges of the pipes and bubbles up to the surface of the aqueous absorbent 22.

The aqueous absorbent 22 may contain various reagents. In some embodiments, the scrubbing solution contains salts or salt complexes, such as carbonate or bicarbonate salts or salt complexes. Without being bound by theory, Applicants believe that CO₂ capture in such scrubbing solutions may proceed by one or more of the following representative reactions:

CO₂+NaOH<−>NaHCO₃

CO₂+NaOH<−>½[Na₂CO₃+H₂O]+½CO₂<−>NaHCO₃

Na₂CO₃+H₂O+CO₂<−>2NaHCO₃

It should be understood that other carbonate or bicarbonate salts and salt complexes may be used, not just sodium based.

In some embodiments, the aqueous absorbent 22 has such a high pH to enhance scrubber performance by quickly absorbing carbon dioxide, such as a pH between about 9 and 11, or between about 10 and 11.

Because CO₂ has a very low solubility in water, dissolving CO₂ in water as a means of transport is impractical. CO₂ solubility in water is about 0.0004, or one pound of CO₂ per ton of water. As such, 1,000,000 pounds of CO₂ per hour emitted from one 700 MW boiler would require 1,000,000 tons per hour or 4,000,000 GPM of water flow. Using water alone as the reagent would require sparging the carbon dioxide at great depths, such as depths of up to six feet. As a result, high fan power would be required to force the flue gas against the higher pressure.

Transporting the CO₂ from the stack to square miles of surface as a concentrated stream of carbonate salt solves this problem. The use of carbonate salt complexes in scrubber reagents allows for the concentration of CO₂ equivalents in mass per gallon of water, so that to reduce the liquid flow between the scrubbers and the photo-bioreactors is greatly reduced. Concentrated sodium bicarbonate solutions can carry one hundred times as much carbon dioxide per gallon as could be dissolved in just water.

The use of sodium bicarbonate provides an additional advantage in that certain algae, such as Spirulina, grow well in concentrated sodium carbonate/sodium bicarbonate solutions. As such, embodiments of the invention therefore employ a scrubber reagent which can also function as a growing media for algae. Therefore, the use of sodium carbonate, or a similar material as a scrubber reagent not only allows absorption of large volumes of carbon dioxide but also can be used to provide a growing medium for certain algae.

A variety of algae and algae mixtures may be used in embodiments of the invention. Suitable algae include, but are not limited to, eukaryotic microalgae and prokaryotic cyanobacteria. Examples of eukaryotic microalgae genera include Chlamydomonas, Nannochloropsis, Dunaliella, Scenedesmus, and Chlorella, and examples of prokaryotic cyanobacterium genera include Aphanizomenon, Spirulina, Synechocystis, and Synechococcus. Combinations of different algal genera, species within a single genus, or combinations of both, can also be used in conjunction with the invention.

The water used in the aqueous absorbent 22 may be ordinary ground water, such as water obtained from a lake, river, or well. When the CO₂ scrubber 16 is used as part of an algae growing system, the aqueous absorbent 22 passes to the algae beds and then recirculates to the CO₂ scrubber 16, with the CO₂ removed by the algae. The system may provide sufficient CO₂ to supply a large bed of algae, such that a very large amount of water may be required for the system, such as 2,000 acre feet for one 600 MW plant. However, because the aqueous absorbent 22 recirculates in the system, little or no water may need to be input into the system once it is operational. However, as the growing algae remove minerals from the water, these minerals may need to be replaced to support continued growth of algae in the aqueous absorbent 22. Water flow through the scrubber should be high to absorb the high concentration of CO₂ in the flue gas. For example, it should allow a once-through flow of water 6 to 12 inches deep to flow constantly through the entire width of the scrubber, which may be, for example, about 100 feet wide in utility scale applications. This flow is much greater than that of SO₂ scrubbers, since CO₂ volume is often ten times the SO₂ volume.

The CO₂ scrubber module 16 of the invention may include a level control for maintaining proper level of aqueous absorbent 22 in the tank 20. The level control may be a second weir 56 extending across the outlet end of the tank, down stream from the partition 26. Aqueous absorbent passes over the weir 56 and to the pump 60. This level control controls the level of the aqueous absorbent 22 between the partition 26 and the weir 56. The aqueous absorbent 22 flows over the second weir 56 and exits the vessel through the fluid outlet 58. It will be noted that the water level on opposite sides of partition 26 are different. The pressure drop across partition 26 may be about 5-15 inches of water depending upon the scrubbing efficiency desired. Flue gas pressure drop must be minimized to reduce fan power requirements. In some embodiments of scrubbers and scrubber systems according to the invention, operations may be carried out with about 6 inches w.g. Utilization of a “primary contactor,” a venturi section for particulate control, is not required.

The presence of a first weir 50 and a second weir 56 on each side of the partition results in multiple levels of aqueous absorbent 22 in vessel 20. The aqueous absorbent 22 entering the scrubber module will be at the highest level. Directly downstream of the first weir 50, the level of the aqueous absorbent 22 will be lower, such as slightly below the lower edge of the partition 26. The aqueous absorbent 22 may be forced lower in this location by higher flue gas pressure created by induced draft fans. Downstream of the partition 26, the level of the aqueous absorbent 22 will increase, such as to about six inches above the lower edge of the partition 26. The level of the aqueous absorbent 22 in this location is controlled by the second weir 56 so that it will be deep enough for CO₂ capture to be adequate. The level of the aqueous absorbent 22 downstream of the second weir 56 will be below the level upstream of this weir, such as several inches lower. The speed of pump 50 can be controlled to maintain this level within a desired range.

Unlike SO₂ wet scrubber systems, the aqueous absorbent 22 is not immediately cycled back into the vessel 22 to be used as a spray. Rather, a pump 60 may continuously take solution from the tank and deliver it through pipes to an algae growing area. For example, the pump may be a conventional centrifugal pump, since algae is not present at the pump location.

The flue gas from boiler 12, having passed through the air quality control system before entering inlet 28 via duct work 14, is at about 130 degrees F., saturated with water vapor. This flue gas enters the tank 20 and is directed into the fingers 32 of the partition 26. The flue gas then passes around the zigzag lower edge of the partition 26 and bubbles upwardly through the aqueous absorbent 22 and out onto the opposite side of the partition 26. Carbon dioxide and sulfur dioxide in the flue gas reacts with the sodium carbonate in the aqueous absorbent 22 to form sodium bicarbonate and sodium sulfate accordance with well known chemical reactions.

Embodiments of the invention may remove between about 70% and 99% of the CO₂ from the flue gas. Some embodiments may remove between about 90% and 99% of the CO₂ from the flue gas. For example, in some embodiments, the flue gas entering the CO₂ scrubber may contain between about 10 and 15% CO₂, and the flue gas exiting the CO₂ scrubber may contain between about 0.1 and 1.5% CO₂. In addition, sulfur dioxide may be removed. For example, 90 to 100% of the SO₂ remaining in the flue gas after passing through the previous SO₂ scrubber may be removed in the CO₂ scrubber.

Scrubber systems having a CO₂ scrubber unit according to embodiments of the invention may include connections to ancillary systems and equipment such as sources for reaction solutions or may be part of larger integrated facility systems and processes. For example, when the CO₂ scrubber unit is in a scrubber system that is part of a larger integrated system, it may be connected to an algae production subsystem or unit that is in fluid communication with the tank of the CO₂ scrubber unit 16 with aqueous absorbent 22 circulating between the CO₂ scrubber 16 and the algae production subsystem. Optionally, for added system flexibility as in the case of serving a cycling industrial load, distributed CO₂ collection systems may serve a central large algae/microbiology growing system. Other features or components may be added to the scrubber system, such as one or more recirculation pumps and storage tanks.

The aqueous absorbent 22, now algae growing media, before entering or after being discharged from the CO₂ scrubber module 16 may pass through a heat exchanger. Heat, normally dissipated in evaporative cooling towers, may be transferred to the algae growing media in this heat exchanger. Algae may thus be furnished with warm growing media for optimum growth rates while the power plant discharges its waste heat without evaporative water loss. To integrate CO₂ scrubbing and algae/microbiology production, the transport medium (solution) functions as both a CO₂ absorber (scrubbing agent) and algae nutrient growing media (solution). In some embodiments, the solution imitates nature, using the CO₂ exchange within sodium carbonate and sodium bicarbonate complex because this is a media in which several algae species naturally grow, such as species of Spirulina. However, there are a plurality of salt solution complexes which may satisfy this dual function. The algae regenerates the solution for use in the CO₂ scrubber without the need for further chemical processing.

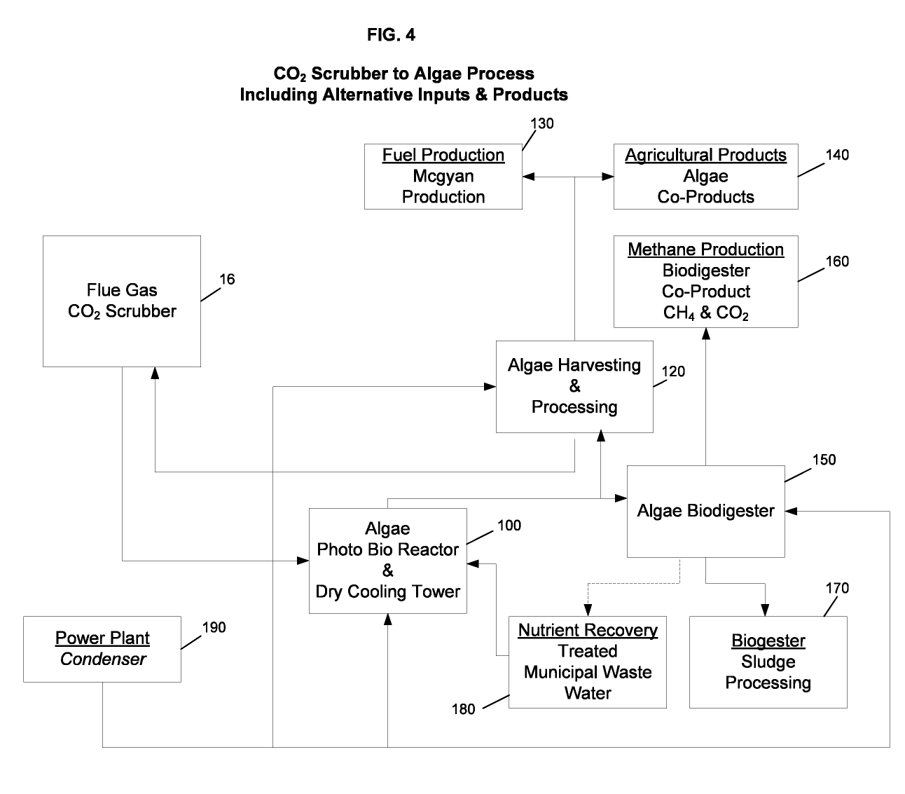

After optionally passing through a heat exchanger, the reagent stream including the absorbent and the CO₂ is directed out to the algae growing area. A schematic diagram of an algae growing system including a CO₂ scrubber is shown in FIG. 4. The CO₂ scrubber 16 is in fluid connection with an algae photo-bioreactor and dry cooling tower 100, thereby supplying the aqueous absorbent to the bioreactors in a form which is heated and rich in CO₂. In some embodiments, the photo-bioreactors may allow algae to be grown all year in either cool or warm latitudes. The algae may be harvested and processed at 120, such as by using filtration, washing and dewatering equipment to separate algae from water (aqueous absorbent 22) and remove residual process salts. The aqueous absorbent is then returned to the CO₂ scrubber 16 and the algae may be used for a variety of end uses that can be in either a wet or dry form.

The algae produced using these systems may be used directly as a fuel in a solid fuel combustion process. Alternatively it may be converted to a fuel in a fuel production system 130, e.g. into transportation fuels such as biodiesel or ethanol. Alternatively, the harvested algae may be used for agricultural products 140 such as animal feed, or soil benefication additives. Other uses include use as a feed stock for industrial and chemical production processes or for coal processes, as a dust control additive, as a source of algal oil, or as a biomass feedstock. Alternatively, the algae may be sent to an algae biodigestor 150 and may be used for methane production 160 for use as gaseous fuel or for sale as natural gas, for processing of biodigestor sludge 170 or for recovery of nutrients 180 which may be returned to the algae bioreactor. In addition, treated municipal waste may provide a source or nutrients to the algae.

The algae bioreactor 100 may utilize a plurality of nutrient feed sources such as effluent from sewage and other waste treatment systems as well as the absorbed CO₂ in the aqueous absorbent 22. Inputs into the algae biodigestor 150 may include many organic wastes, as well as heat from the power plant 190.

Algae growing systems including a CO₂ scrubber, such as the system shown in FIG. 4, may use large water volumes and flue gas flow rates. However, they may function using a low energy consumption. For example, water pumping work may be minimized. Unlike some conventional scrubber systems, the CO₂ scrubber systems do not require the use of spray pumps to enhance liquid-to-gas contact surface ratio. This saves electric power and capital expense. In addition, the scrubber utilizes only a few feet of height, meaning a small or relative short or shallow reaction tank can be used (as compared to SO₂ scrubbers), minimizing structural costs, and provide a high liquid-to-gas contact surface to provide effective capture of target pollutants, e.g., CO₂, by the solution.

In addition to supplying the algae with captured CO₂, the algae growing systems according to embodiments of the invention also supply the algae bioreactors with heat. This heat in the flue gas may be transferred to the bioreactor 100, providing some of the heat required for maintaining the optimum algae growing conditions. In addition, excess heat produced during warmer months may be used to heat the underlying ground, which in turn may heat the solution during colder winter months, creating a geothermal energy storage aspect. Using the scrubber solution to carry heat into the algae growing systems, e.g., bioreactors or ponds, eliminates the need for separate pumping system(s). This combined thermal energy support scheme can advantageously be utilized to maintain growing temperatures during periods when ambient temperatures drop for sustained periods, i.e., winter months and cold nights. For example, the temperature may be maintained above about 80° F. or about 85° F. for certain strains of algae.

A heat exchanger may be used for transferring heat to the algae. Such heat exchangers may include rows of copper tubes across a water flow path. The water inside the tubes might be the algae media growing solution returning from the algae filters. (It would not contain algae, except a small fraction that passes through the filters.) The water flow on the outside of the tubes might be the cooling water that normally is pumped to the evaporative cooling towers. The heat exchanger may be located upstream of the CO₂ scrubber. This placement would be advantageous in summer weather, to maximize water conservation, because at this location the solution is at its lowest temperature, so it can cool the water flowing back to the condenser to a lower temperature. A low temperature here provides a desirable, low condensing temperature for the steam in the condenser. This results in a more efficient power generating system since the steam turbine can expand the steam to a lower pressure (because the temperature of the steam is lower). Alternatively, the heat exchanger may be located downstream of the CO₂ scrubber 16, in which case it will operate at a higher temperature because the aqueous absorbent 22 will be warmed in the scrubber 16 as it condenses flue gas water vapor.

Heat may also be drawn from thermal waste streams. The CO₂ scrubbing process cools the flue gas warming the common solution for the scrubber—algae photo-bioreactor. The effluent from process cooling water cycles, such as a power plant turbine-generator condenser may be diverted from either cooling tower heat rejection cycles or from open stream discharge and may be used to add additional heat to the common system solution used in the algae photo-bioreactor. Using these heat sources to create a combined heat and generation process not only improves the thermal efficiency but also captures and conserves large volumes of water, which is critically important in arid locales.

Water used in the system may be conserved, such as by condensing between about 50% and about 67% of the water vapor in the flue gas. In addition, algae dewatering, drying and/or integral end uses may be configured to condense and recycle most or essentially all water extracted from the process, with the algae using water to grow.

The CO₂ scrubber 16 according to embodiments of the invention can provide several other advantages. The scrubber 16 uses a very large flow of reagent through the tank to accommodate the relatively high concentration of carbon dioxide in the flue gas (such as about 11 to 13%) compared, for instance, to the concentration of sulfur dioxide in the flue gas, which may be about 0.5 to 3%. Conventional scrubbers with spray tower designs would require large pumping systems to circulate the reagent through the flue gas. Power costs would be excessive. Using reagent streams previously cooled in the algae growing area allows the reagent streams to enter the scrubber module at relatively low temperature, about 85 to 95° F. Flue gas passing through the cool solution is quenched so that more than 50% of the water vapor in the flue gas condenses into the scrubber liquid. This conserves large quantities of valuable water that is later used by the algae. Carbon dioxide is effectively captured due to the high liquid-to-gas ratio surrounding the gas bubbles as the gas passes up through the aqueous absorbent 22.

Other advantages include lower cost to manufacture compared to conventional SO₂ scrubbers. In addition, the CO₂ scrubber does not require pumps to directly recycle the scrubbing solution back into the scrubber and has a minimal flue gas pressure drop, with the pressure drop through the liquid downstream of the partition being supplied by induced draft fans. It also provides a means for large liquid flow through the scrubber and can maintains the scrubbing solution at a low temperature that facilitates capture of water vapor from the flue gas (quenching the flue gas to nominally 100° F.).

In alternative embodiments, the CO₂ could be gathered from flue gas generated by surrounding generators, for example. The CO₂ could then be sent to a location near a nuclear power plant for use in an algae farm. If the nuclear plant is required to conserve water, the dry cooling of the algae farm could conserve water for the power plant, while also disposing of the CO₂ from the other sources.

Although particular embodiments of the invention have been illustrated in the accompanying Drawings and described herein, it will be understood that the invention is not limited only to the embodiments disclosed, but is intended to embrace any alternatives, equivalents, modifications, and/or rearrangement of elements falling within the scope of the invention as defined by the following claims.

What is claimed:

1. A scrubber unit, comprising:
a first chamber having a gas inlet and a fluid inlet;
a second chamber having a gas outlet and a fluid outlet; and
a partition dividing the first chamber and the second chamber, wherein the partition is positioned to allow fluid to flow from the first chamber to the second chamber beneath a lower edge of the partition;
a first weir to distribute the flow across the first chamber
a second weir in the second chamber.

2. The scrubber of claim 1 wherein the first weir extends across a width of the first chamber such that fluid entering the first chamber through the fluid inlet must flow over the first weir to pass through the scrubber.

3. The scrubber of claim 1 wherein the second weir is sized such that a desired fluid level is maintained between the partition and the second weir.

4. The scrubber of claim 1 further comprising a aqueous absorbent, wherein the aqueous absorbent flows into the scrubber through the fluid inlet and out of the scrubber through the fluid outlet.

5. The scrubber of claim 4 wherein the aqueous absorbent comprises a carbonate salt and a bicarbonate salt.

6. The scrubber of claim 5 wherein the aqueous absorbent comprises sodium carbonate and sodium bicarbonate.

7. The scrubber of claim 6 wherein the pH of the aqueous absorbent is between about 9 and 11.

8. The scrubber of claim 1 wherein the lower edge of the partition forms a zigzag.

9. The scrubber of claim 1 wherein the scrubber removes between about 70 and 99% of the carbon dioxide from the flue gas.

10. An algae growing system comprising:
a wet scrubber for removal of carbon dioxide from flue gas;
an aqueous absorbent salt solution; and
an algae bioreactor;
wherein the aqueous absorbent solution flows in a continuous circuit through the wet scrubber and the algae bioreactor to absorb carbon dioxide in the wet scrubber and to provide the absorbed carbon dioxide to algae.

11. The algae growing system of claim 10 wherein the aqueous absorbent comprises a carbonate salt and a bicarbonate salt.

12. The algae growing system of claim 11 wherein the aqueous absorbent comprises sodium carbonate and sodium bicarbonate.

13. The algae growing system of claim 12 wherein the algae comprises a species of Spirulina.

14. The algae growing system of claim 10 further comprising a heat exchanger, wherein the aqueous absorbent flows from the scrubber to the heat exchanger, and then from the heat exchanger to the algae bioreactor.

15. A method of removing carbon dioxide from flue gas comprising:
passing flue gas through a gas inlet into first chamber of a scrubber, the first chamber including a fluid inlet and an aqueous absorbent in a lower portion of the first chamber;
bubbling the flue gas into the aqueous absorbent;
flowing the aqueous absorbent including the flue gas bubbles into a lower portion of a second chamber of the scrubber;
releasing the flue gas bubbles as clean flue gas from the aqueous absorbent and into the second chamber; and
passing the gas through a gas outlet of the second chamber.

16. The method of claim 15 wherein the first chamber includes a weir, further comprising flowing the aqueous absorbent over a first weir to distribute the aqueous absorbent across the width of the first chamber.

17. The method of claim 16 wherein the second chamber includes a weir, further comprising controlling a depth of aqueous absorbent between the partition and the second weir.

18. The method of claim 15 wherein the aqueous absorbent comprises a carbonate salt and a bicarbonate salt.

19. The method of claim 18 wherein the aqueous absorbent comprises sodium carbonate and sodium bicarbonate.

20. The method of clam 15 further comprising removing between about 70 and 99% of the carbon dioxide from the flue gas.
